(19) United States
(12) Patent Application Publication
(10) Pub. No.: US 2021/0232954 A1
(43) Pub. Date: Jul. 29, 2021

(54) PREDICTIVE DATA ANALYSIS USING CUSTOM-PARAMETERIZED DIMENSIONALITY REDUCTION

(71) Applicant: Optum Services (Ireland) Limited, Dublin (IE)

(72) Inventors: David S. Monaghan, Dublin (IE); Megan O'Brien, Dublin (IE); Kenneth Bryan, Dublin (IE); Chirag Chadha, Dublin (IE)

(21) Appl. No.: 16/750,475

(22) Filed: Jan. 23, 2020

(51) Int. Cl. G06N 5/04; G06K 9/62; G16B 40/00; G16B 20/20

(52) U.S. Cl. CPC G06N 5/045; G16B 20/20; G16B 40/00; G06K 9/6215

(57) ABSTRACT

There is a need for more effective and efficient predictive data analysis. This need can be addressed by, for example, solutions for performing/executing predictive data analysis using custom-parameterized dimensionality reduction. In one example, a method includes identifying a group of predictive input features and one or more predictive markers; determining a per-marker feature for each predictive marker; determining one or more refined features for the group of predictive input features based at least in part on each per-marker feature for a predictive marker; performing the predictive inference based at least in part on the one or more refined features to generate one or more predictions; and performing one or more prediction-based actions based at least in pat on the one or more predictions.

### BACKGROUND

Various embodiments of the present invention address technical challenges related to performing predictive data analysis. Various embodiments of the present invention address the shortcomings of existing predictive inference systems and disclose various techniques for efficiently and reliably performing predictive data analysis.

### BRIEF SUMMARY

In general, embodiments of the present invention provide methods, apparatus, systems, computing devices, computing entities, and/or the like for performing predictive data analysis using custom-parameterized dimensionality reduction. Certain embodiments utilize systems, methods, and computer program products that predictive data analysis using custom-parameterized dimensionality reduction by utilizing one or more predictive markers, per-marker proximate subsets, per-marker features, predictive distance measures, predictive geometric spectrums, predictive spectrum units, and predictive correlation analyses.

In accordance with one aspect, a method is provided. In one embodiment, the method comprises identifying a group of predictive input features, wherein each predictive input feature is associated with an input feature position in a predictive geometric spectrum; identifying one or more predictive markers, wherein each predictive marker is associated with a marker position in the predictive geometric spectrum; for each predictive marker: (i) determining a per-marker proximate subset of the group of predictive input features for the predictive marker based at least in part on the marker position for the predictive marker and each input feature position for a predictive input feature of the group of predictive input features, (ii) determining, for each predictive input feature in the per-marker proximate subset, a per-feature correlation value for the predictive input feature and a target feature associated with the predictive inference, and (iii) determining, based at least in part on each per-feature correlation value for a predictive input feature in the per-marker proximate subset, a per-marker feature for the predictive marker; determining one or more refined features for the group of predictive input features based at least in part on each per-marker feature for a predictive marker of the one or more predictive markers; performing the predictive inference based at least in part on the one or more refined features to generate one or more predictions; and performing one or more prediction-based actions based at least in part on the one or more predictions.

In accordance with another aspect, a computer program product is provided. The computer program product may comprise at least one computer-readable storage medium having computer-readable program code portions stored therein, the computer-readable program code portions comprising executable portions configured to identify a group of predictive input features, wherein each predictive input feature is associated with an input feature position in a predictive geometric spectrum; identify one or more predictive markers, wherein each predictive marker is associated with a marker position in the predictive geometric spectrum; for each predictive marker: (i) determine a per-marker proximate subset of the group of predictive input features for the predictive marker based at least in part on the marker position for the predictive marker and each input feature position for a predictive input feature of the group of predictive input features, (ii) determine, for each predictive input feature in the per-marker proximate subset, a per-feature correlation value for the predictive input feature and a target feature associated with the predictive inference, and (iii) determine, based at least in part on each per-feature correlation value for a predictive input feature in the per-marker proximate subset, a per-marker feature for the predictive marker; determine one or more refined features for the group of predictive input features based at least in part on each per-marker feature for a predictive marker of the one or more predictive markers; perform the predictive inference based at least in part on the one or more refined features to generate one or more predictions; and perform one or more prediction-based actions based at least in part on the one or more predictions.

In accordance with yet another aspect, an apparatus comprising at least one processor and at least one memory including computer program code is provided. In one embodiment, the at least one memory and the computer program code may be configured to, with the processor, cause the apparatus to identify a group of predictive input features, wherein each predictive input feature is associated with an input feature position in a predictive geometric spectrum; identify one or more predictive markers, wherein each predictive marker is associated with a marker position in the predictive geometric spectrum; for each predictive marker: (i) determine a per-marker proximate subset of the group of predictive input features for the predictive marker based at least in part on the marker position for the predictive marker and each input feature position for a predictive input feature of the group of predictive input features, (ii) determine, for each predictive input feature in the per-marker proximate subset, a per-feature correlation value for the predictive input feature and a target feature associated with the predictive inference, and (iii) determine, based at least in part on each per-feature correlation value for a predictive input feature in the per-marker proximate subset, a per-marker feature for the predictive marker; determine one or more refined features for the group of predictive input features based at least in part on each per-marker feature for a predictive marker of the one or more predictive markers; perform the predictive inference based at least in part on the one or more refined features to generate one or more predictions; and perform one or more prediction-based actions based at least in part on the one or more predictions.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive inference computing entity in accordance with some embodiments discussed herein.

FIG. 3 provides an example external computing entity in accordance with some embodiments discussed herein.

FIG. 4 is a flowchart diagram of an example process for performing predictive inference using custom-parameterized dimensionality reduction in accordance with some embodiments discussed herein.

FIG. 5 provides an operational example of a correlation plot data object in accordance with some embodiments discussed herein.

FIG. 6 is a flowchart diagram of an example process for determining a per-marker feature for a predictive marker in accordance with some embodiments discussed herein.

FIG. 7 is a flowchart diagram of an example process for determining a per-feature correlation value for a predictive input feature and a target feature in accordance with some embodiments discussed herein.

FIG. 8 provides an operational example of a zygosity value data object in accordance with some embodiments discussed herein.

FIG. 9 provides an operational example of a categorical per-marker feature calculation data object in accordance with some embodiments discussed herein.

FIG. 10 provides an operational example of a numerical per-marker feature calculation data object in accordance with some embodiments discussed herein.

FIG. 11 is a flowchart diagram of an example process for determining refined features for a predictive marker in accordance with some embodiments discussed herein.

### DETAILED DESCRIPTION

Various embodiments of the present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. OVERVIEW

Dimensionality reduction is the practice of reducing the number of raw input features used for predictive data analysis by mapping each raw input feature to one or more refined features. By utilizing dimensionality reduction, predictive data analysis systems are often able to enhance the efficiency and accuracy of their training processes and inference processes. Various embodiments of the present invention introduce dimensionality reduction techniques that are more efficient and more reliable than state-of-the-art dimensionality reduction techniques for various applications. In doing so, various embodiments of the present invention enhance the efficiency and accuracy of existing predictive data analysis systems and make important technical contributions to the field of predictive data analysis.

In some predictive domains, predictive input features have complex inter-feature relationships that may undermine the utility of utilizing existing predictive dimensionality reduction techniques to perform dimensionality reduction on such predictive input features. For example, the predictive input features may be exceedingly numerous, be related to distinct real-world super-structures, and often have marginal individual predictive significance while having greater predictive significance when interacting with other predictive input features. An example of such a complex predictive domain is a genomic predictive domain, where tens of thousands of genetic variants related to thousands of genes can be relevant to performing genomic-related predictive inferences. Moreover, individual genetic variants may each have marginal individual significance for performing some particular genomic predictive analyses but nevertheless have substantial collective significance when analyzed in interaction with other genetic variants for performing the noted particular genomic predictive analyses.

Observations of the inventors and their algorithmic analyses of various existing dimensionality reduction techniques show that such dimensionality reduction techniques fail to efficiently and effectively perform dimensionality reduction in the complex predictive domains described above. For example, a prevalent dimensionality reduction technique known as Principal Component Analysis (PCA) has many shortcomings that undermine the ability of PCA solutions to perform effective and efficient dimensionality reduction in complex prediction domains. For example, PCA solutions often fail to enable developers to integrate domain-level information (e.g., information about associations of particular genes or other biological structures with a particular target feature) into the dimensionality reduction process. As another example, PCA solutions often fail to analyze interactions of different combinations of features in performing dimensionality reduction routines. Instead, PCA solutions often largely consolidate features based at least in part on their co-variance, without considering predictive significance of particular feature combinations and without taking class labels (e.g., gene labels, chromosome labels, biological pathway labels, biological complex labels, and/or the like) of predictive input features into account.

Various embodiments of the present invention address the above-noted shortcomings of existing dimensionality reduction techniques by introducing techniques for custom-parameterized dimensionality reduction. In some embodiments, the custom-parameterized dimensionality reduction techniques described herein can be utilized to integrate predictive domain information in selecting combinations of raw input features utilized to analyze the effect of inter-feature interactions on predictive outcomes. For example, in some embodiments, the custom-parameterized dimensionality reduction techniques utilize results of latest genomic research identifying particular genes having the most correlation with a target feature to combine particular genetic variants deemed sufficiently related to the particular genes in order to analyze inter-variant interactions of the noted genetic variants. The noted embodiments can then utilize the predictive insights about predictive significance of various inter-variant interactions of genetic variants deemed sufficiently related to genes of interest to generate refined features in an efficient and effective manner. In doing so, various embodiments of the present invention are able to enhance the efficiency and accuracy of various predictive data analysis systems being utilized in complex prediction domains.

### II. COMPUTER PROGRAM PRODUCTS, METHODS, AND COMPUTING ENTITIES

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### III. EXEMPLARY SYSTEM ARCHITECTURE

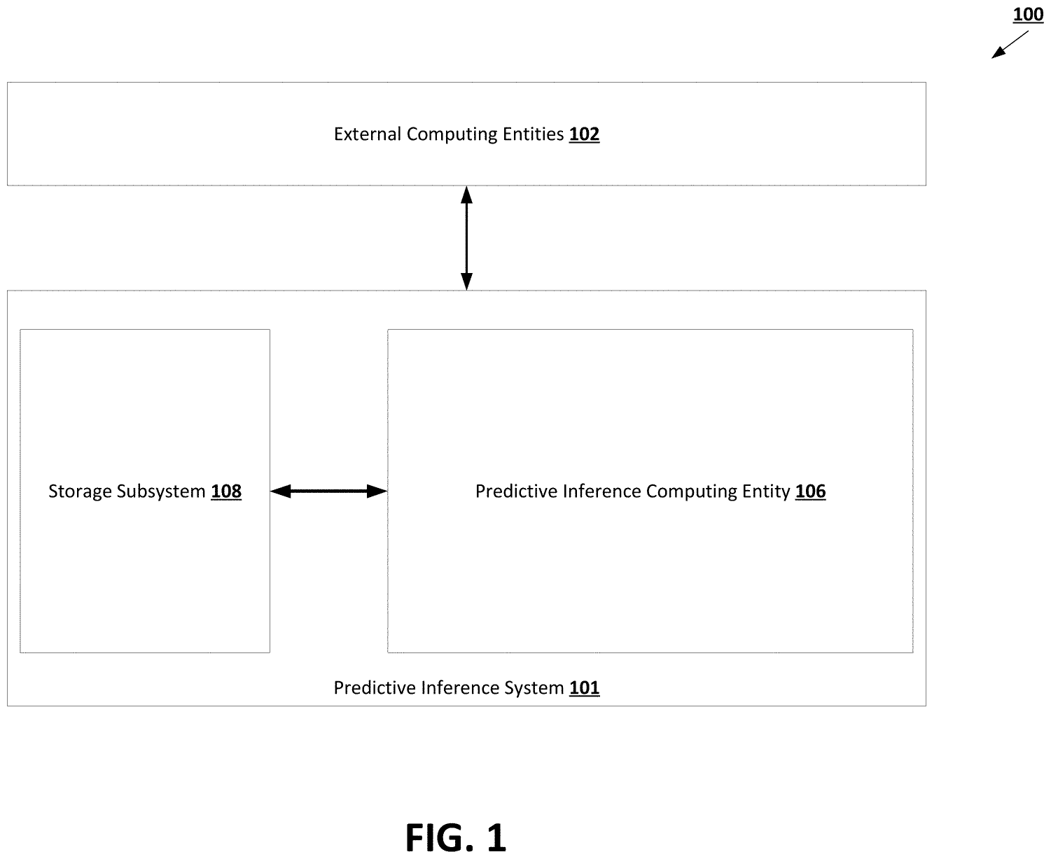

FIG. 1 is a schematic diagram of an example architecture 100 for performing predictive data analysis using custom-parameterized dimensionality reduction. The architecture 100 includes a predictive inference system 101 configured to receive predictive data analysis requests from external computing entities 102, process the predictive data analysis requests to generate predictions, provide the generated predictions to the external computing entities 102, and automatically perform prediction-based actions based at least in part on the generated predictions. An example of a predictive data analysis task is generating health-related predictions based at least in part on genetic input data associated with a patient and performing prediction-based actions based on the generated health-related predictions.

In some embodiments, predictive inference system 101 may communicate with at least one of the external computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive inference system 101 may include a predictive inference computing entity 106 and a storage subsystem 108. The predictive inference computing entity 106 may be configured to receive predictive data analysis requests from one or more external computing entities 102, process the predictive data analysis requests to generate the generated predictions corresponding to the predictive data analysis requests, provide the generated predictions to the external computing entities 102, and automatically perform prediction-based actions based at least in part on the generated predictions.

The storage subsystem 108 may be configured to store input data used by the predictive inference computing entity 106 to perform predictive data analysis as well as model definition data used by the predictive inference computing entity 106 to perform various predictive data analysis tasks. The storage subsystem 108 may further store underlying real-world measurement data and/or underlying real-world observation data used to determine per-feature correlation values and per-marker correlation values as part of performing predictive data analysis using custom-parameterized dimensionality reduction. The storage subsystem 108 may further store information about how to perform automated prediction-based actions based on particular generated predictions.

The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Inference Computing Entity

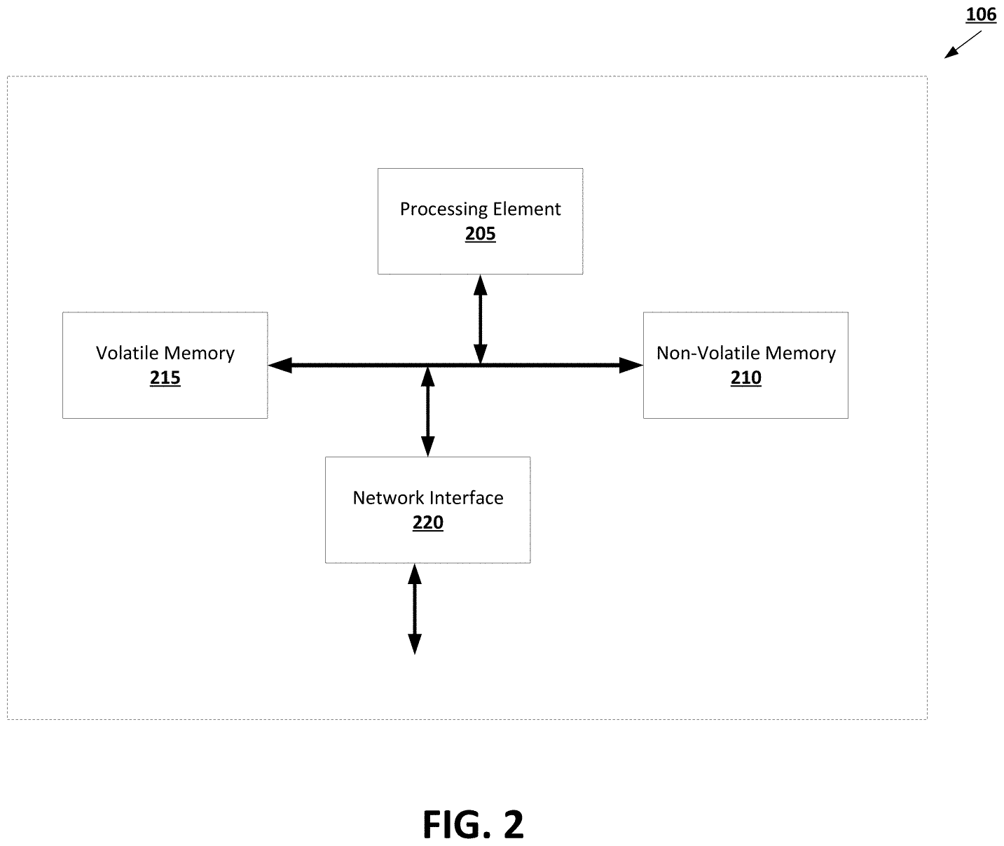

FIG. 2 provides a schematic of a predictive inference computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive inference computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive inference computing entity 106 may include or be in communication with one or more processing elements 205 (also referred to as processors, processing circuitry, and/or similar terms used herein interchangeably) that communicate with other elements within the predictive inference computing entity 106 via a bus, for example. As will be understood, the processing element 205 may be embodied in a number of different ways. For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive inference computing entity 106 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the predictive inference computing entity 106 may further include or be in communication with volatile media (also referred to as volatile storage, memory, memory storage, memory circuitry and/or similar terms used herein interchangeably). In one embodiment, the volatile storage or memory may also include one or more volatile storage or memory media 215, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive inference computing entity 106 with the assistance of the processing element 205 and operating system.

As indicated, in one embodiment, the predictive inference computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive inference computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive inference computing entity 106 may include or be in communication with one or more input elements, such as a keyboard input, a mouse input, a touch screen/display input, motion input, movement input, audio input, pointing device input, joystick input, keypad input, and/or the like. The predictive inference computing entity 106 may also include or be in communication with one or more output elements (not shown), such as audio output, video output, screen/display output, motion output, movement output, and/or the like.

### Exemplary External Computing Entity

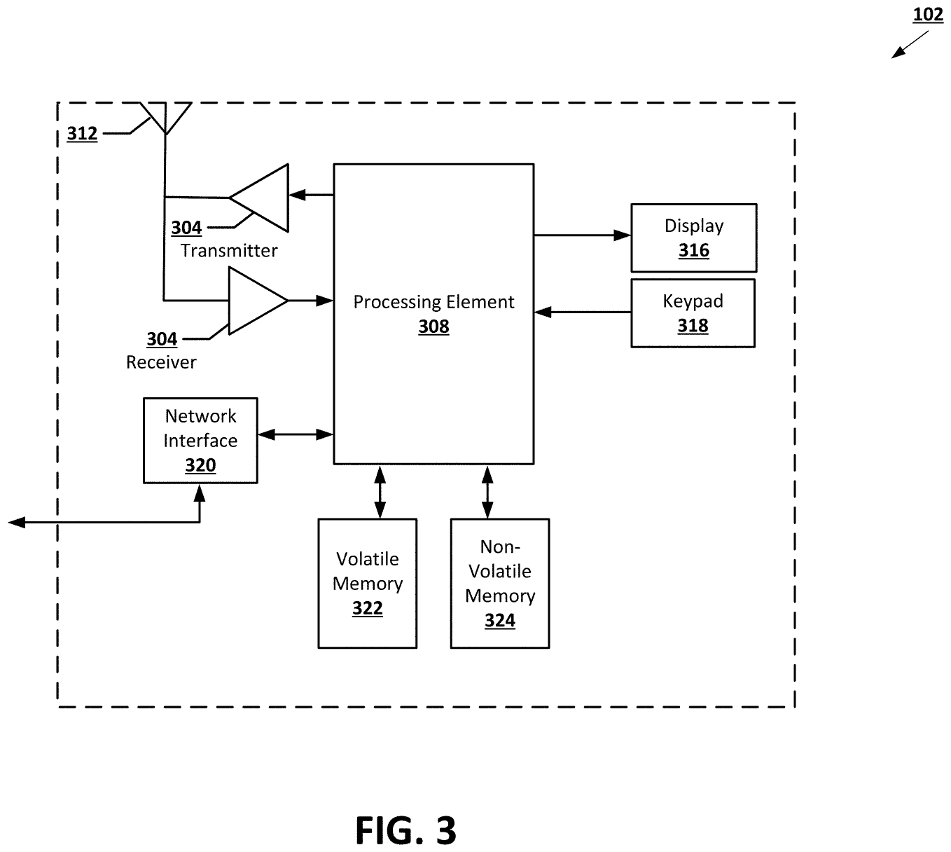

FIG. 3 provides an illustrative schematic representative of an external computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. External computing entities 102 can be operated by various parties. As shown in FIG. 3, the external computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the external computing entity 102 may be capable of operating with one or more air interface standards, communication protocols, modulation types, and access types. More particularly, the external computing entity 102 may operate in accordance with any of a number of wireless communication standards and protocols, such as those described above with regard to the predictive inference computing entity 106. In a particular embodiment, the external computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102 may operate in accordance with multiple wired communication standards and protocols, such as those described above with regard to the predictive inference computing entity 106 via a network interface 320.

Via these communication standards and protocols, the external computing entity 102 can communicate with various other entities using concepts such as Unstructured Supplementary Service Data (USSD), Short Message Service (SMS), Multimedia Messaging Service (MIMS), Dual-Tone Multi-Frequency Signaling (DTMF), and/or Subscriber Identity Module Dialer (SIM dialer). The external computing entity 102 can also download changes, add-ons, and updates, for instance, to its firmware, software (e.g., including executable instructions, applications, program modules), and operating system.

According to one embodiment, the external computing entity 102 may include location determining aspects, devices, modules, functionalities, and/or similar words used herein interchangeably. For example, the external computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the external computing entity's 102 position in connection with a variety of other systems, including cellular towers, Wi-Fi access points, and/or the like. Similarly, the external computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The external computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the external computing entity 102 to interact with and/or cause display of information/data from the predictive inference computing entity 106, as described herein. The user input interface can comprise any of a number of devices or interfaces allowing the external computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the external computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The external computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the external computing entity 102. As indicated, this may include a user application that is resident on the entity or accessible through a browser or other user interface for communicating with the predictive inference computing entity 106 and/or various other computing entities.

In another embodiment, the external computing entity 102 may include one or more components or functionality that are the same or similar to those of the predictive inference computing entity 106, as described in greater detail above. As will be recognized, these architectures and descriptions are provided for exemplary purposes only and are not limiting to the various embodiments.

In various embodiments, the external computing entity 102 may be embodied as an artificial intelligence (AI) computing entity, such as an Amazon Echo, Amazon Echo Dot, Amazon Show, Google Home, and/or the like. Accordingly, the external computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### IV. EXEMPLARY SYSTEM OPERATIONS

Dimensionality reduction is the practice of reducing the number of raw input features used for predictive data analysis by mapping each raw input feature to one or more refined features. By utilizing dimensionality reduction, predictive data analysis systems are often able to enhance the efficiency and accuracy of their training processes and inference processes. Various embodiments of the present invention introduce dimensionality reduction techniques that are more efficient and more reliable than state-of-the-art dimensionality reduction techniques for various applications. In doing so, various embodiments of the present invention enhance the efficiency and accuracy of existing predictive data analysis systems and make important technical contributions to the field of predictive data analysis.

Various embodiments of the present invention address the above-noted shortcomings of existing dimensionality reduction techniques by introducing techniques for custom-parameterized dimensionality reduction. In some embodiments, the custom-parameterized dimensionality reduction techniques described herein can be utilized to integrate predictive domain information in selecting combinations of raw input features utilized to analyze the effect of inter-feature interactions on predictive outcomes. For example, in some embodiments, the custom-parameterized dimensionality reduction techniques utilize results of latest genomic research identifying particular genes having the most correlation with a target feature to combine particular genetic variants deemed sufficiently related to the particular genes in order to analyze inter-variant interactions of the noted genetic variants. The noted embodiments can then utilize the predictive insights about predictive significance of various inter-variant interactions of genetic variants deemed sufficiently related to genes of interest to generate refined features in an efficient and effective manner. In doing so, various embodiments of the present invention are able to enhance the efficiency and accuracy of various predictive data analysis systems being utilized in complex prediction domains.

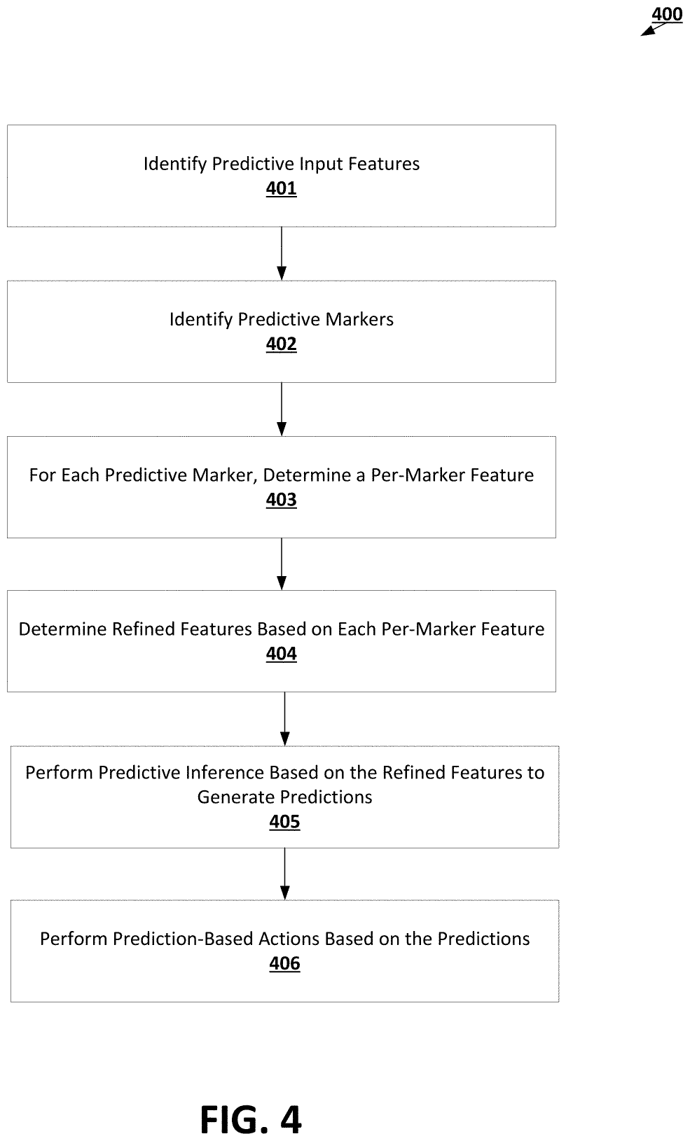

FIG. 4 is a flowchart diagram of an example process 400 for performing predictive inference using custom-parameterized dimensionality reduction. Via the various steps/operations of FIG. 4, a predictive inference computing entity 106 can utilize domain-specific insights to generate domain-aware refined predictive features based at least in part on raw predictive features, thus in turn increasing efficiency and reliability of predictive data analysis in complex predictive domains.

The process 400 begins at step/operation 401 when the predictive inference computing entity 106 identifies a group of predictive input features, wherein each predictive input feature is associated with an input feature position in a predictive geometric spectrum. A predictive input feature may be any data object describing a raw predictive feature. For example, a predictive input feature may describe a categorical raw predictive feature (e.g., an ordinal categorical raw predictive feature) or a numeric raw predictive feature.

A categorical raw predictive feature is a raw predictive feature that can assume one of many potential predictive feature values and where the precise distance between the potential predictive feature values is deemed numerically unknown. An example of a categorical raw predictive feature is a predictive feature that describes size of a real-world object (e.g., a t-shirt) as one of a set of ordinal categories (e.g., small, medium, large, extra-large, and/or the like), where a set of ordinal categories refer to two or more categories that can be ordered or ranked (as opposed to a set of nominal categories that cannot be ordered or ranked). Other examples of raw categorical predictive features may include postal-code-describing predictive features, size-describing predictive features, predictive features that describe zygosities of single-nucleotide polymorphisms (SNPs) in individuals, and/or the like.

In contrast, a numeric raw predictive feature is a raw predictive feature that that can assume one of many potential predictive feature values and where the precise distance between the potential predictive feature values is deemed numerically known. Examples of raw numeric predictive features may include height-describing predictive features, weight-describing predictive features, age-describing predictive features, heart-rate-describing predictive features, and/or the like.

In some embodiments, a predictive geometric spectrum is a data object that defines a group of geometric positions as well as a predictive distance measure between each pair of geometric positions. In some embodiments, predictive input features and/or predictive markers can each be mapped to a geometric position of the group of geometric positions defined by the predictive geometric spectrum, such that the geometric distances between the mapped geometric positions of the predictive input features and/or the predictive markers can then be utilized to determine predictive distance measures between pairs of predictive input features, pairs of predictive markers, and/or feature-marker pairs comprising a predictive input feature and a predictive marker.

In some embodiments, the predictive geometric spectrum defines one or more predictive spectrum units each comprising a subset of the group of geometric positions defined by the predictive spectrum unit. In some of those embodiments, if a first predictive input feature and/or a first predictive marker is mapped to a first geometric position that is in a different predictive spectrum unit than a predictive spectrum unit of a second geometric position of a second predictive input feature and/or a second predictive marker, then the predictive distance measure between the noted predictive input features and/or predictive markers is deemed to have an a excessively large value and/or a maximal value, e.g., an infinity value, such as a positive infinity value.

In some embodiments, each predictive input feature in the group of predictive input features is associated with a genetic variant, such as an SNP. In some embodiments, the predictive geometric spectrum defines a geometric distance between each pair of mapped SNPs. In some embodiments, the predictive geometric spectrum defines one or more predictive spectrum units each associated with a grouping of SNPs, such as with a chromosome-based grouping of SNPs. In some embodiments, each predictive input feature in the group of predictive input features is associated with a numeric feature type, such as height, weight, age, and/or the like.

In some embodiments, the predictive geometric spectrum defines one or more one or more predictive spectrum units each associated with a grouping of numeric feature types, such as with a grouping of numeric feature types deemed related to a particular genetic unit such as a chromosome and/or a grouping of numeric feature types deemed related to a higher-level feature type. In some embodiments, SNPs can be grouped together based on other biological characteristics (e.g., protein pathways) to form predictive spectrum units.

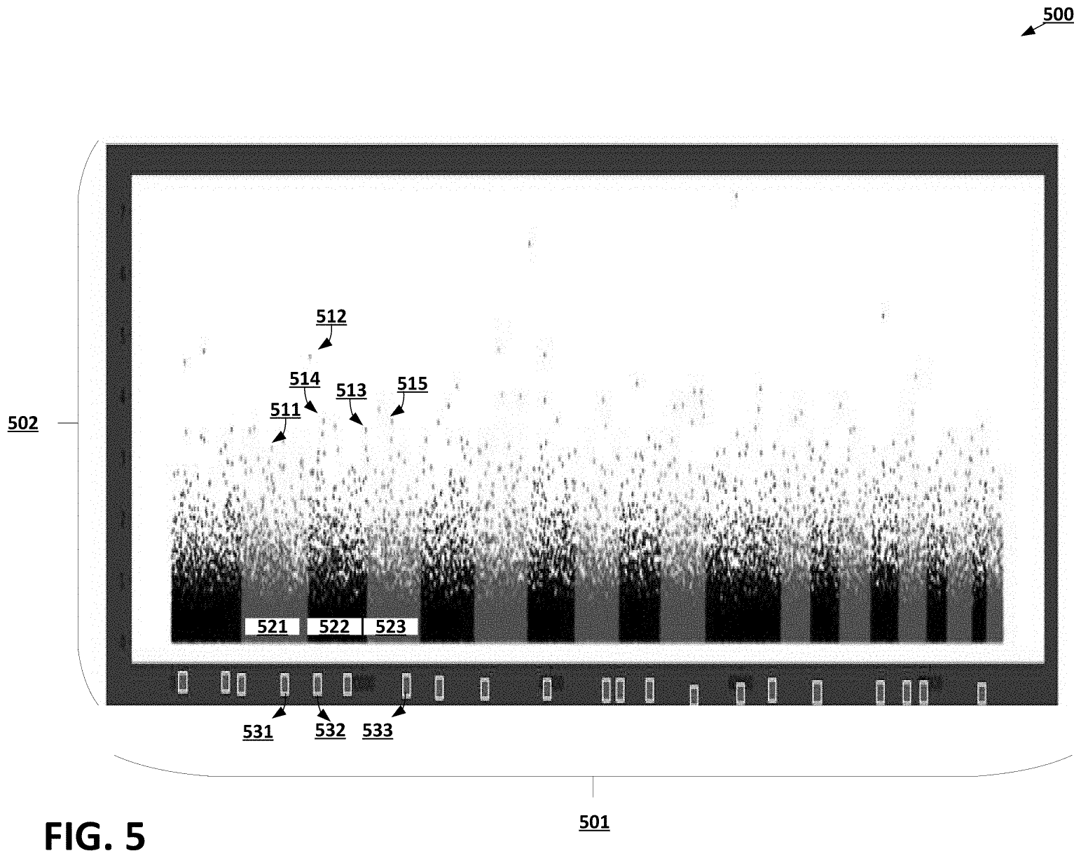

An operational example of a predictive geometric spectrum 501 is provided in FIG. 5, which is an operational example of a correlation plot data object 500 of various genetic variants, where the genetic variants are examples of predictive input features as described herein. As depicted in FIG. 5, the correlation plot data object 500 includes, in addition to a predictive geometric spectrum 501 indicated by its horizontal axis data, a per-feature correlation spectrum 502 indicated by its vertical axis data. In some embodiments, the vertical axis data indicate an inverted version of the per-feature correlation spectrum 502, such that higher correlations are at the top of the vertical axis of the per-feature correlation spectrum 502 and lower correlations are at a bottom of the vertical axis of the per-feature correlation spectrum 502.

As further depicted in the correlation plot data object 500, each point of the plot relates to a respective genetic variant and depicts a per-feature correlation variant of the respective genetic variant. For example, point 511 depicts a per-feature correlation variant of a respective first genetic variant, point 512 depicts a per-feature correlation variant of a respective second genetic variant, point 513 depicts a per-feature correlation variant of a respective third genetic variant, point 514 depicts a per-feature correlation variant of a respective fourth genetic variant, and point 515 depicts a per-feature correlation variant of a respective fifth genetic variant.

As further depicted in FIG. 5, the predictive geometric spectrum 501 of the correlation plot data object 500 is divided into various predictive spectrum units each associated with a chromosome. For example, predictive spectrum unit 521 includes genetic variants associated with a first chromosome, such as the first genetic variant. As another example, predictive spectrum unit 522 includes genetic variants associated with a second chromosome, such as the second genetic variant, the third genetic variant, and the fourth genetic variant. As a further example, predictive spectrum unit 523 includes genetic variants associated with a third chromosome, such as the fifth genetic variant.

In some embodiments, in accordance with the predictive geometric spectrum 501 of the correlation plot data object 500, the predictive inference computing entity 106 can determine that the following pairs of genetic variants have a maximal predictive distance value because they lie in different predictive spectrum units: the first genetic variant and the second genetic variant, the first genetic variant and the third genetic variant, the first genetic variant and the fourth genetic variant, the first genetic variant and the fifth genetic variant, the fifth genetic variant and the second genetic variant, the fifth genetic variant and the third genetic variant, and the fifth genetic variant and the fourth genetic variant.

In some embodiments, in accordance with the predictive geometric spectrum 501 of the correlation plot data object 500, the predictive inference computing entity 106 can determine that the following pairs of genetic variants have a non-maximal predictive distance value because they lie in the same predictive spectrum unit: the second genetic variant and the third genetic variant, the second genetic variant and the fourth genetic variant, and the third genetic variant and the fourth genetic variant.

In some embodiments, in accordance with the predictive geometric spectrum 501 of the correlation plot data object 500, the predictive inference computing entity 106 can determine that the predictive distance measure between the second genetic variant and the fourth genetic variant is greater than the predictive distance between the third genetic variant and the fourth genetic variant because the geometric distance between the second genetic variant and the fourth genetic variant is larger than the geometric distance between the third genetic variant and the fourth genetic variant. In some embodiments, in accordance with the predictive geometric spectrum 501 of the correlation plot data object 500, the predictive inference computing entity 106 can determine that the predictive distance measure between the second genetic variant and the fourth genetic variant is equal to than the predictive distance between the third genetic variant and the fourth genetic variant because all three noted genetic variants belong to the same predictive spectrum unit, i.e., predictive spectrum unit 522.

Returning to FIG. 4, at step/operation 402, the predictive inference computing entity 106 identifies one or more predictive markers, wherein each predictive marker is associated with a marker position in the predictive geometric spectrum. In some embodiments, a predictive marker is a data object that describes a higher-level feature, e.g., a higher-level feature determined based at least in part on predictive domain data to have likely strong correlation with a target feature. For example, a predictive marker may describe a higher-level feature determined based on a gene and/or other biological feature (e.g., a biological pathway, a gene complex, and/or the like). As another example, a predictive marker may describe a higher-level feature determined based on one SNP or a collection of two or more SNPs.

A higher-level feature may describe a categorical higher-level feature (e.g., an ordinal categorical higher-level feature) or a numeric higher-level feature. A categorical higher-level predictive feature is a higher-level predictive feature that can assume one of many potential predictive feature values and where the precise distance between the potential predictive feature values is deemed numerically unknown. An example of a categorical higher-level predictive feature is a predictive feature that describes a gene deemed closest to a particular gene. An ordinal categorical higher-level predictive feature is a categorical higher-level predictive feature which is associated with a set of potential categories that can be ordered or ranked.

In contrast, a numeric higher-level predictive feature is a higher-level predictive feature that that can assume one of many potential predictive feature values and where the precise distance between the potential predictive feature values is deemed numerically known. An example of a numeric higher-level predictive feature is a predictive feature that describes likely contribution of a particular gene to a particular physical condition and/or bodily feature. Another example of a numeric higher-level predictive feature is a predictive feature that describes a likely effectiveness of a drug to addressing a particular physical condition given correlation value of the SNPs of a particular to the particular physical condition.

Returning to FIG. 5, the predictive geometric spectrum 501 of the correlation plot data object 500 defines marker positions for various predictive markers. For example, the predictive geometric spectrum 501 of the correlation plot data object 500 defines the marker position 531 for a first predictive marker, the marker position 532 for a second predictive marker, and the marker position 533 for a third predictive marker.

In some embodiments, in accordance with the predictive geometric spectrum 501 of the correlation plot data object 500, the predictive inference computing entity 106 can determine that the first predictive marker has a maximal predictive distance from the second predictive input feature associated with the point 512, the third predictive input feature associated with the point 513, the fourth predictive input feature associated with the point 514, and the fifth predictive input feature associated with the point 515, because the first predictive marker is in the first predictive spectrum unit 521 while the noted predictive input features are not in the first predictive spectrum unit 521.

As another example, in accordance with the predictive geometric spectrum 501 of the correlation plot data object 500, the predictive inference computing entity 106 can determine that the second predictive marker has a maximal predictive distance from the first predictive input feature associated with the point 511 and the fifth predictive input feature associated with the point 515 because the second predictive marker is in the second predictive spectrum unit 522 while the noted predictive input features are not in the second predictive spectrum unit 522.

As a further example, in accordance with the predictive geometric spectrum 501 of the correlation plot data object 500, the predictive inference computing entity 106 can determine that the fifth predictive marker has a maximal predictive distance from the first predictive input feature associated with the point 511, the second predictive input feature associated with the point 512, the third predictive input feature associated with the point 513, and the fourth predictive input feature associated with the point 514 because the fifth predictive marker is in the third predictive spectrum unit 523 while the noted predictive input features are not in the third predictive spectrum unit 523.

In some embodiments, if a predictive input feature and a predictive marker are in the same predictive spectrum unit of a predictive geometric spectrum, the predictive inference computing entity 106 can utilize a geometric distance between an input feature position for the predictive input feature and a marker position for the predictive marker to determine the feature-marker distance between the predictive input feature and the predictive marker. For example, in accordance with the predictive geometric spectrum 501 of the correlation plot data object 500, the predictive inference computing entity 106 can determine that the second predictive marker has a smaller predictive distance measure relative to the second predictive input feature relative to the third predictive input feature because the geometric distance between the second predictive marker and the second predictive input feature is smaller than the geometric distance between the second predictive marker and the third predictive input feature.

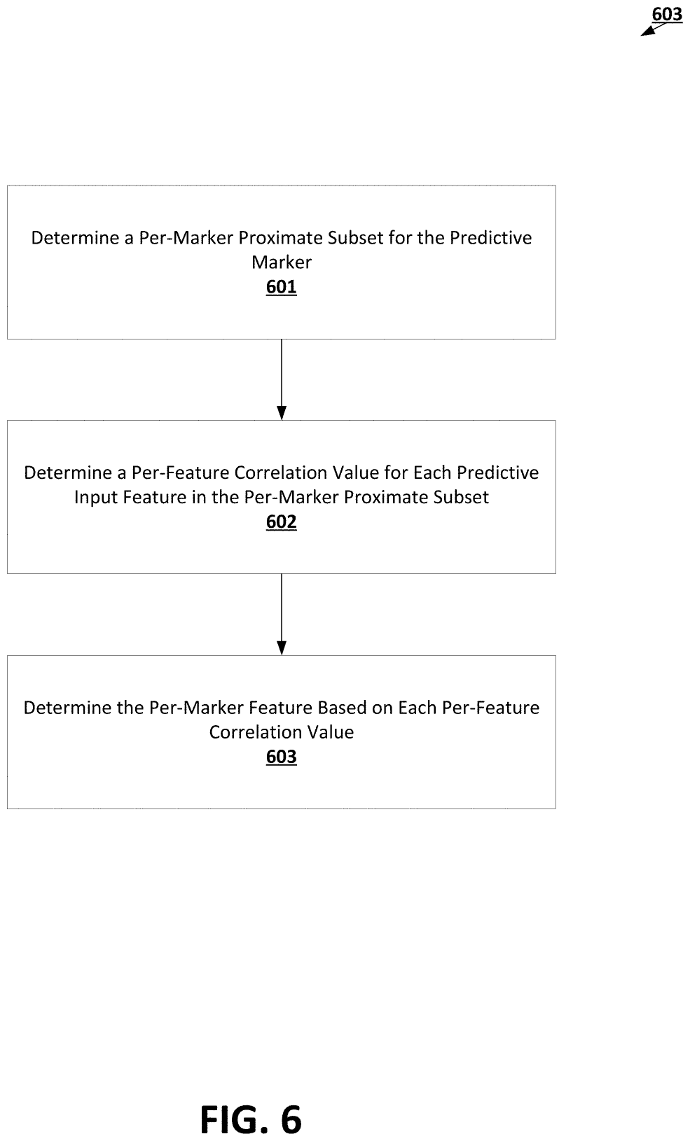

Returning to FIG. 4, at step/operation 403, the predictive inference computing entity 106 determines a per-marker feature for each predictive marker identified in step/operation 402. In some embodiments, step/operation 403 can be performed in accordance with the steps/operations depicted in FIG. 6, which is a flowchart diagram of an example process for determining a per-marker feature for a predictive marker. The process depicted in FIG. 6 begins at step/operation 601 when the predictive inference computing entity 106 determines a per-marker proximate subset of the group of predictive input features for the predictive marker based at least in part on the marker position for the predictive marker and each input feature position for a predictive input feature of the group of predictive input features.

In some embodiments, the predictive inference computing entity 106 determines that a predictive input feature is in the per-marker proximate subset for a predictive marker if the input feature position for the predictive input feature is within the same predictive spectrum unit as the marker position for the predictive marker. In some embodiments, the predictive inference computing entity 106 determines that a predictive input feature is in the per-marker proximate subset for a predictive marker if a geometric distance of the input feature position for the predictive input feature and the marker position for the predictive marker as determined based at least in part on the predictive geometric spectrum is below a threshold geometric distance of the predictive marker. In some embodiments, the predictive inference computing entity 106 determines that a predictive input feature is in the per-marker proximate subset for a predictive marker if both of the following conditions are met: (i) the input feature position for the predictive input feature is within the same predictive spectrum unit as the marker position for the predictive marker, and (ii) a geometric distance of the input feature position for the predictive input feature and the marker position for the predictive marker as determined based at least in part on the predictive geometric spectrum is below a threshold geometric distance of the particular predictive marker.

In some embodiments, to determine a per-marker proximate subset of the group of predictive input features for the predictive marker, the predictive inference computing entity 106 determines, for each predictive input feature in the group of predictive input features, a feature-marker predictive distance measure in the predictive geometric spectrum between the predictive input feature and the predictive marker associated with predictive marker; and determines the per-marker proximate subset for the predictive marker based at least in part on each feature-marker predictive distance measure for a predictive input feature in the group of predictive input features. In some of the noted embodiments, the predictive geometric spectrum defines one or more predictive spectrum units, the one or more predictive spectrum units comprise a target predictive spectrum unit for the predictive marker, and the feature-marker predictive distance measure for a predictive input feature in the group of predictive input features is set to a maximal value if the input feature position for the predictive input feature falls outside the target predictive spectrum unit.

At step/operation 602, the predictive inference computing entity 106 determines, for each predictive input feature in the per-marker proximate subset determined in step/operation 601, a per-feature correlation value between the predictive input feature and a target feature associated with the predictive inference. In some embodiments, the per-feature correlation value between a predictive input feature and a target feature is a data object that describes an estimated contribution of values adopted by the predictive input feature to detecting the target feature. For example, the per-feature correlation value for a particular predictive input feature associated with a genetic variant (e.g., an SNP) may describe an association of a zygosity value of the genetic variant in a genome of a particular individual and a target feature describing the gene deemed most similar to the gene associated with the genetic variant. As another example, the per-feature correlation value for a particular predictive input feature associated with a genetic variant (e.g., an SNP) may describe an association of a zygosity value of the genetic variant in a genome of a particular individual and a target feature describing predicted hair color of an individual. As a further example, the per-feature correlation value for a particular predictive input feature associated with a raw numeric feature may describe an association of the raw numeric feature and an effectiveness of a drug for an individual associated with the raw numeric feature.

In some embodiments, if both the predictive input feature and the target feature relate to categorical features, the per-feature correlation value for the predictive input feature and the target feature is an association value that describes a log of odds ratio for the predictive input feature and the particular feature. In some embodiments, if at least one of the predictive input feature and the target feature relate to numerical features, the per-feature correlation value for the predictive input feature and the target feature is a Pearson coefficient value. In some embodiments, if at least one of the predictive input feature and the target feature relate to ordinal categorical features, the per-feature correlation value for the predictive input feature and the target feature is a Spearman's rank correlation coefficient.

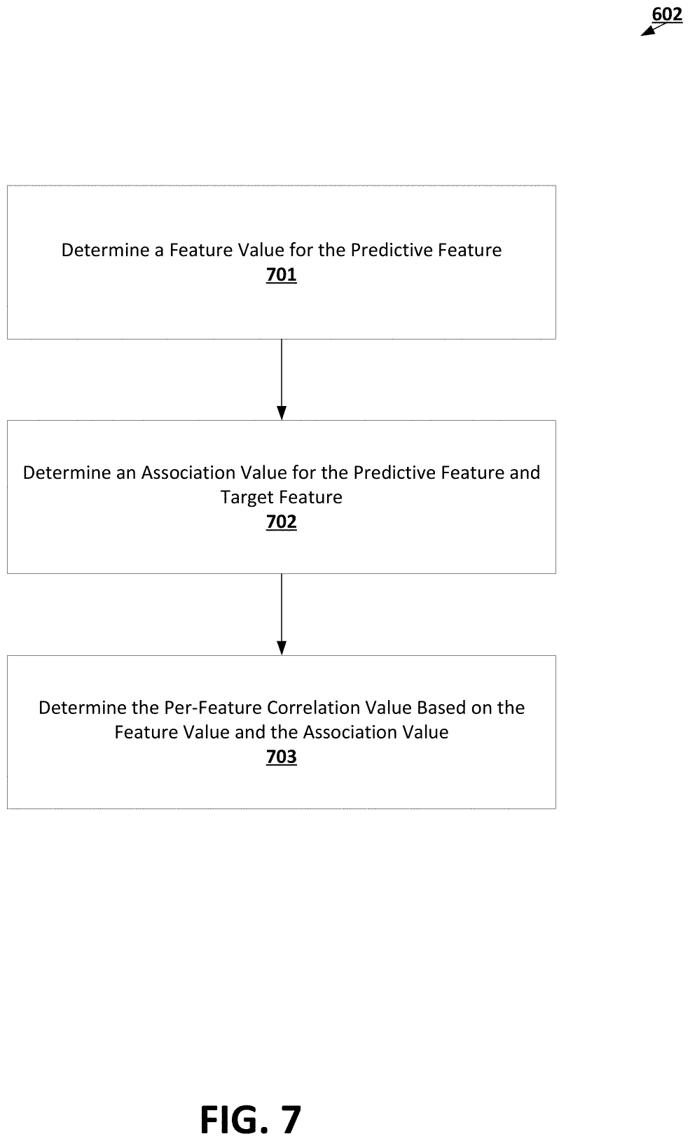

In some embodiments, step/operation 602 may be performed in accordance with the process depicted in FIG. 7, which is a flowchart diagram of an example process for determining a per-feature correlation value for a predictive input feature and a target feature. The process depicted in FIG. 7 begins at step/operation 701 when the predictive inference computing entity 106 determines a feature value for the predictive input feature, e.g., the measured and/or observed value of the predictive feature described by the predictive input feature in a predictive scenario.

For example, the feature value for a particular predictive input feature may describe the zygosity value of a particular SNP in a particular individual. In some embodiments, the feature value for a particular predictive input feature associated with a particular SNP may have a first value (e.g., a value of zero) if the SNP has a homozygous reference in an individual, a second value (e.g., a value of one) if the SNP has a heterozygous variation in an individual, and a third value (e.g., a value of two) if the SNP has a homozygous variation in an individual. As another example, the feature value for a particular predictive input feature may describe the height value of a particular individual.

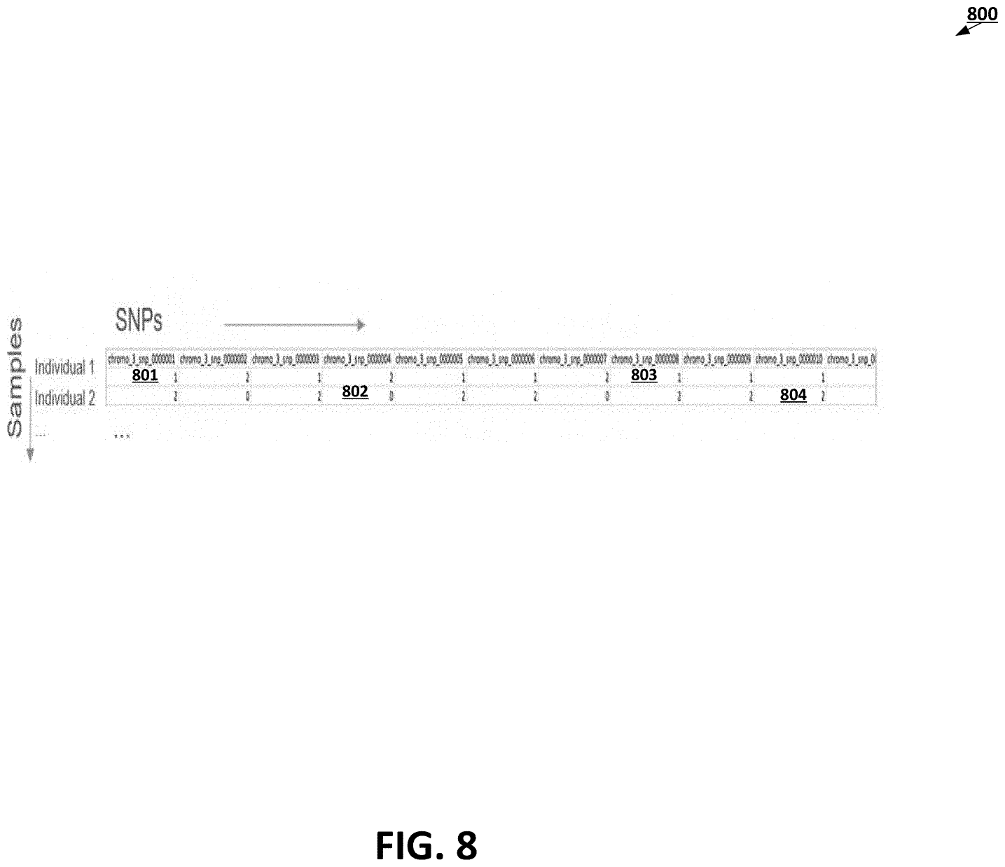

An operational example of zygosity values for predictive input features associated with various SNPs is presented in the zygosity value data object 800 of FIG. 8. As depicted in FIG. 8, the zygosity value data object 800 includes a zygosity value for each SNP in a particular individual.

For example, as indicated by the zygosity value 801 in the zygosity value data object 800, the first SNP related to a third chromosome in a first individual is associated with a zygosity indicated by the number one. As another example, as indicated by the zygosity value 802 in the zygosity value data object 800, the fourth SNP related to a third chromosome in individual 2 is associated with a zygosity indicated by the number zero. As yet another example, as indicated by the zygosity value 803 in the zygosity value data object 800, the eighth SNP related to a third chromosome in a first individual is associated with a zygosity indicated by the number one. As a further example, as indicated by the zygosity value 804 in the zygosity value data object 800, the tenth SNP related to a third chromosome in individual 2 is associated with a zygosity indicated by the number two.

Returning to FIG. 7, at step/operation 702, the predictive inference computing entity 106 determines an association value (e.g., a statistical association value) for the predictive input feature and the target feature. The association value may describe any measure of association between the predictive input feature and the target feature. Examples of the noted association measures for a predictive input feature and a target feature include an odds ratio for the predictive input feature and the target feature, a log of odds ratio for the predictive input feature and the target feature, a Pearson correlation coefficient ratio for the predictive input feature and the target feature, a Spearman's rank correlation coefficient for the predictive input feature and the target feature, and/or the like. In some embodiments, to determine the log of odds ratios for a predictive input feature and a target feature, the predictive inference computing entity 106 takes a defined log (e.g., the natural log) of an odds ratio for the predictive input feature and the target feature.

In some embodiments, to generate the odds ratio for the predictive input feature and the target feature, the predictive inference computing entity 106 first divides the number of cases of individuals having the predictive feature associated with predictive input feature who show the target feature by the number of cases of individuals having the predictive feature associated with predictive input feature who fail to show the target feature to generate an affirmative odds value. Afterward, the predictive inference computing entity 106 divides the number of cases of individuals not having the predictive feature associated with predictive input feature who show the target feature by the number of cases of individuals not having the predictive feature associated with predictive input feature who fail to show the target feature to generate a negative odds value. Next, the predictive inference computing entity 106 divides the affirmative odds value by the negative odds value to generate the odds ratio. Thereafter, the predictive inference computing entity 106 can take the natural log of the odds ratio to generate an association measure for the predictive input feature and the target feature.

For example, consider Table 1 presented below that shows alphabetical labels for the number of individuals having a particular SNP in their genome who show a particular target feature (i.e., the label a), the number of individuals having a particular SNP in their genome who fail show a particular target feature (i.e., the label b), the number of individuals not having a particular SNP in their genome who show a particular target feature (i.e., the label c), the number of individuals not having a particular SNP in their genome who fail show a particular target feature (i.e., the label d):

TABLE 1

| | . . . Responder to | . . . Non-Responder to |
|---|---|---|
| | Feature B | Feature B |
| # of Cases | a | b |
| With SNP A | | |
| and . . . | | |
| # of Cases | c | d |
| With SNP A | | |
| and . . . | | |

In some embodiments, given the above-presented Table 1, the log of odds ratio of the predictive input feature associated with the SNP A and the target feature associated with the feature B may be determined using the equation

$$ln ( \frac{n}{m} ) , where n = \frac{a}{b} and m = \frac{c}{d} .$$

At step/operation 703, the predictive inference computing entity 106 determines the per-feature correlation value for the predictive input feature based at least in part on the feature value determined in step/operation 702 and the association value determined in step/operation 702. In some embodiments, the predictive inference computing entity 106 multiplies the feature value for the predictive input feature and the association value for the predictive input feature with respect to the target feature to determine the per-feature correlation value for the feature value with respect to the target feature.

Returning to FIG. 6, at step/operation 603, the predictive inference computing entity 106 determines, based at least in part on each per-feature correlation value for a predictive input feature in the per-marker proximate subset of the predictive marker, a per-marker feature for the predictive marker. In some embodiments, the predictive inference computing entity 106 generates a measure of statistical distribution (e.g., a mean, median, mode, sum, and/or the like) of each per-feature correlation value for a predictive input feature in the per-marker proximate subset of the predictive marker and determines the per-marker feature for the predictive marker based at least in part on the generated measure of statistical distribution. In some embodiments, to determine the per-marker feature for the predictive marker, the predictive inference computing entity 106 utilizes the operations described by the below Equation 1:

$$f ( x , y ) = \frac{{ρ}_{{x}_{1} , y } {x}_{1} + {ρ}_{{x}_{2} , y} {x}_{2} + … + {ρ}_{{x}_{n} , y} {x}_{n}}{n} \qquad Equation 1$$

In some of the embodiments utilizing the operations described in Equation 1, f(x,y) is the per-marker feature for the predictive marker, xᵢ is the feature value for the ith predictive input feature in the per-marker proximate subset for the predictive marker, pₓ_{ᵢ}_{,y is the Pearson correlation coefficient for the ith predictive input feature and the target feature y, and n is the number of predictive input features in the the per-marker proximate subset of the predictive marker.}

In some embodiments, to determine the per-marker feature for the predictive marker, the predictive inference computing entity 106 utilizes the operations described by the below Equation 2:

$$f ( x , y ) = \frac{L {x}_{1} + L {x}_{2} + … + {L}_{{x}_{n} , y} {x}_{n}}{n} \qquad Equation 2$$

In some of the embodiments utilizing the operations described in Equation 2, f(x,y) is the per-marker feature for the predictive marker, xᵢ is the feature value for the ith predictive input feature in the per-marker proximate subset for the predictive marker, Lₓ_{ᵢ}_{,y}, is the log of odds ratio for the ith predictive input feature and the target feature y, and n is the number of predictive input features in the the per-marker proximate subset of the predictive marker.

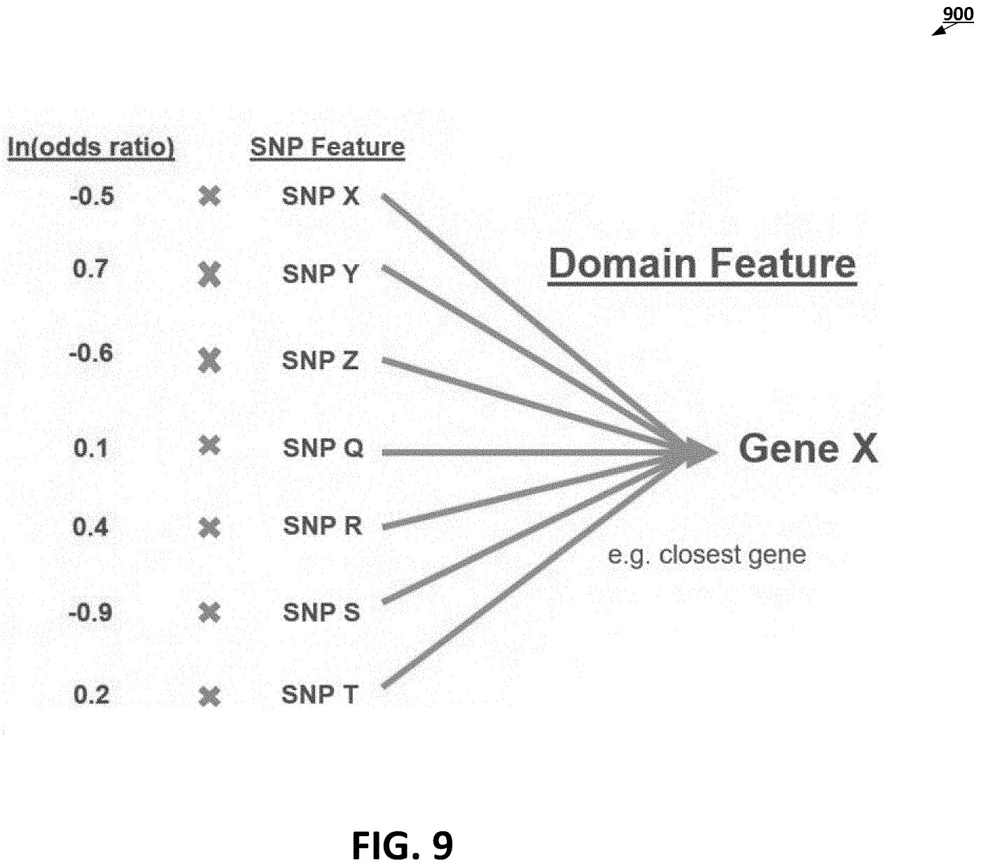

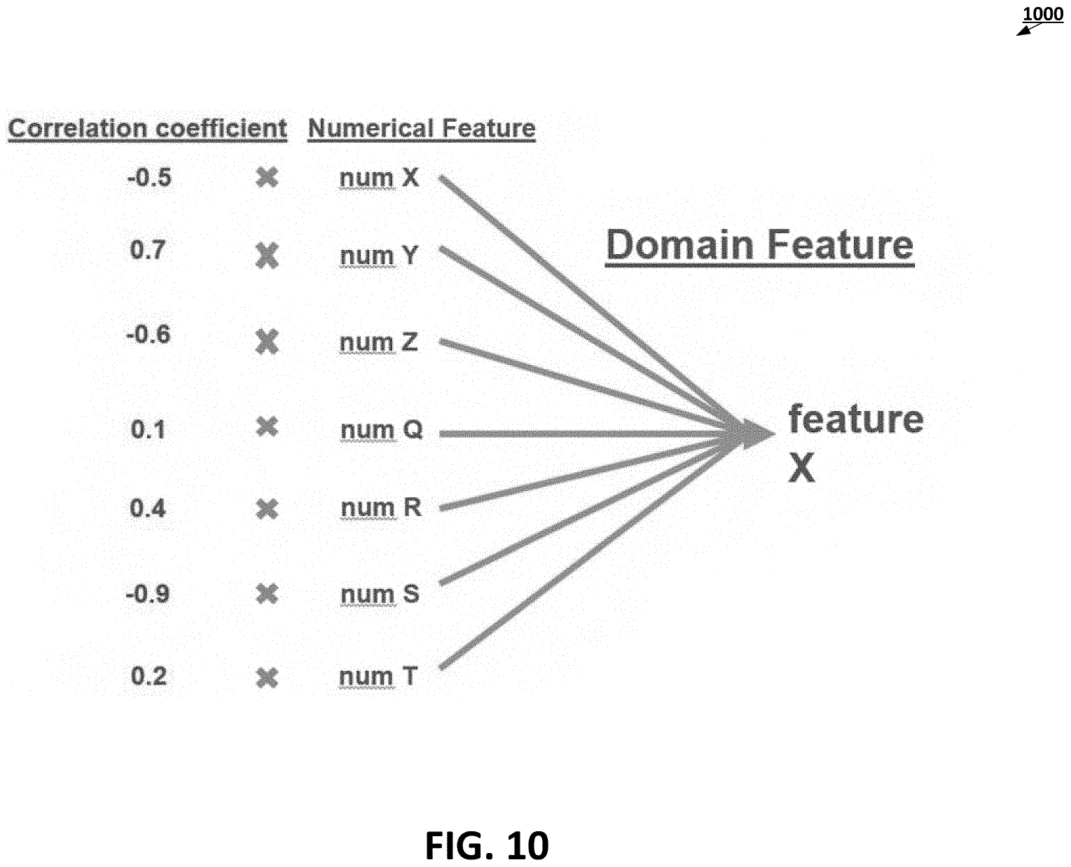

Operational examples of generating per-marker features are depicted in FIGS. 9-10. FIG. 9 depicts an ordinal categorical per-marker feature calculation data object 900 that generates per-marker features based at least in part on input values derived from multiplying SNP values associated with SNPs deemed related to a gene of interest to log of odds ratios associated with the noted SNPs that are deemed related to the gene of interest. FIG. 10 depicts a numeric per-marker feature calculation data object that generates per-marker features based at least in part on input values derived from multiplying numerical feature values for numeric input features deemed related to a higher-level feature of interest with correlation coefficients (e.g., Pearson correlation coefficients) for the numeric input features deemed related to the noted higher-level feature of interest.

Returning to FIG. 4, at step/operation 404, the predictive inference computing entity 106 determines one or more refined features for the group of predictive input features identified in step/operation 401 based at least in part on each per-marker feature for a predictive marker of the one or more predictive markers. In some embodiments, the predictive inference computing entity 106 adopts each per-marker feature for a predictive marker of the one or more predictive markers as a refined feature of the one or more refined features. In some embodiments, the predictive inference computing entity 106 adopts a per-marker feature that is associated a predictive marker as a refined feature if a per-marker correlation value for the per-marker feature in relation to a target feature exceeds all of the per-feature correlation values for the predictive input features in the per-marker proximate subset of the predictive marker in relation to the target feature.

In some embodiments, the predictive inference computing entity 106 adopts a per-marker feature that is associated a predictive marker as a refined feature if a per-marker correlation value for the per-marker feature exceeds a measure of statistical distribution (e.g., a mean, weighted mean, median, mode, standard deviation, and/or the like) of the per-feature correlation values for the predictive input features in the per-marker proximate subset of the predictive marker.

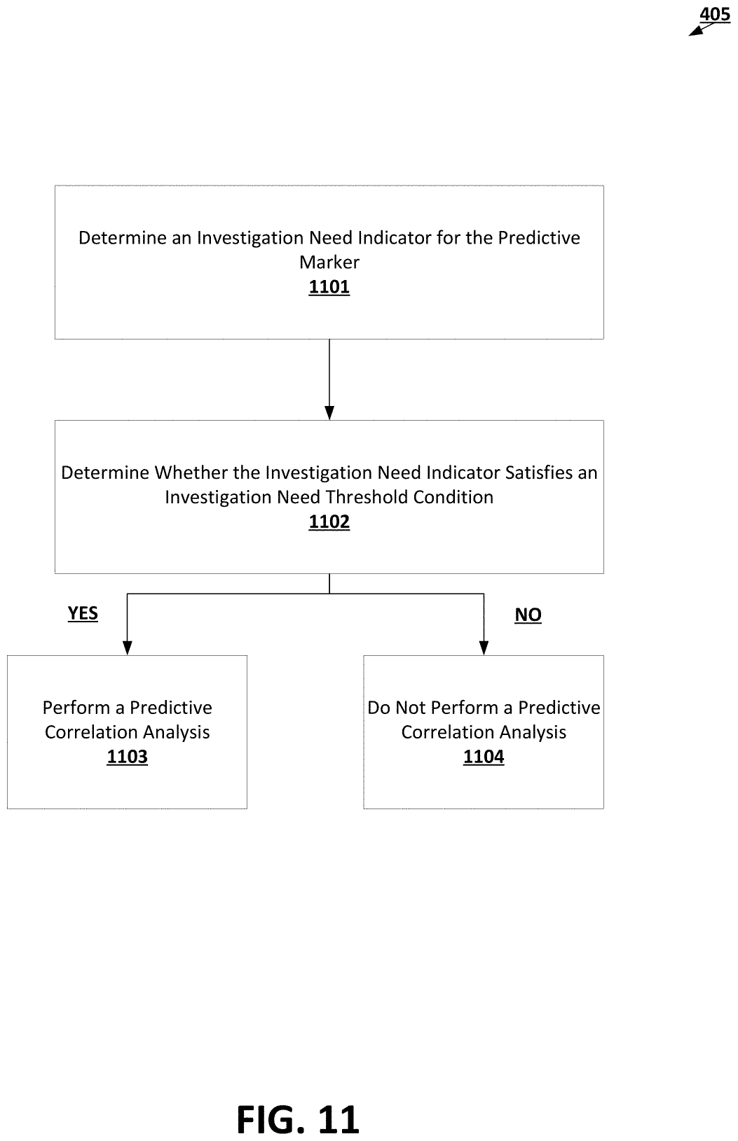

In some embodiments, step/operation 404 may be performed in accordance with the process depicted in FIG. 11, which is an operational example of a flowchart diagram of an example process for determining refined features for a predictive marker based at least in part on the per-marker feature for the predictive marker. The process depicted in FIG. 11 begins at step/operation 1101 when the predictive inference computing entity 106 determines an investigation need indicator for the predictive indicator based at least in part on a per-marker correlation value for the per-marker feature associated with the predictive feature and each per-feature correlation value for a related predictive input feature of one or more related predictive input features associated with the predictive marker. In some embodiments, the one or more related predictive input features associated with the predictive marker include each predictive input feature in the group of predictive input features that belongs to the per-marker proximate subset for the predictive marker.

In some embodiments, the predictive inference computing entity 106 determines the investigation need indicator for the predictive marker based at least in part on whether per-marker correlation value for the per-marker feature exceeds all of the per-feature correlation values associated with the one or more related predictive input features associated with the predictive marker. In some embodiments, the predictive inference computing entity 106 determines the investigation need indicator for the predictive marker based at least in part on whether per-marker correlation value for the per-marker feature exceeds a measure of statistical distribution of the per-feature correlation values associated with the one or more related predictive input features associated with the predictive marker. In some embodiments, the investigation need indicator is a binary value. In some embodiments, the investigation need indicator is a continuous numeric value. In some embodiments, the investigation need indicator is a discrete numeric value.

At step/operation 1102, the predictive inference computing entity 106 determines whether the investigation need indicator satisfies an investigation need threshold condition. In some embodiments, the predictive inference computing entity 106 determines that the investigation need indicator satisfies the investigation need threshold condition if the investigation need indicator indicates a need for investigating predictive significance of interactions between at least one combination of two or more of the one or more related predictive input features associated with the predictive marker.

At step/operation 1103, in response to determining that the investigation need indicator satisfies the investigation need threshold condition, the predictive inference computing entity 106 performs a predictive correlation analysis on the one or more related predictive input features to determine a related subset of the one or more refined features. In some embodiments, in response to determining that the investigation need indicator satisfies the investigation need threshold condition, the predictive correlation analysis is configured to detect one or more inter-subset correlations for the predictive marker, where each inter-subset correlation may indicate a conclusion about predictive significance of interaction of two or more corresponding predictive input features of the one or more related predictive input features associated with the predictive marker in predicting the target feature. In some of the noted embodiments, the predictive correlation analysis is configured to determine a refined predictive feature for each inter-subset correlation based at least in part on feature values and association values of the related predictive input features associated with the inter-subset correlation.

In some embodiments, in response to determining that the investigation need indicator satisfies the investigation need threshold condition, the predictive inference computing entity 106 analyzes whether interactions of various groupings of two or more predictive input features of the one or more related predictive input features associated with the predictive marker have predictive significance. If the predictive inference computing entity 106 determines that the interactions of a particular grouping of two or more predictive input features has predictive significance, the predictive inference computing entity 106 combines the two or more predictive input features in the particular grouping in order to generate a corresponding refined feature for the group of predictive input features identified in step/operation 401.

At step/operation 1104, in response to determining that the investigation need indicator fails to satisfy the investigation need threshold condition, the predictive inference computing entity 106 does not perform a predictive correlation analysis on the one or more related predictive input features. In some embodiments, in response to determining that the investigation need indicator fails to satisfy the investigation need threshold condition, the predictive inference computing entity 106 adopts the one or more related predictive input features associated with the predictive marker as refined features.

Returning to FIG. 4, at step/operation 405, the predictive inference computing entity 106 performs the predictive inference based at least in part on the one or more refined features determined in step/operation 404 to generate one or more predictions. In some embodiments, the predictive inference computing entity 106 processes the one or more predictions using a machine learning model (e.g., a machine learning model utilizing a neural network, an unsupervised machine learning model, a Bayesian network machine learning model, and/or the like) to generate the one or more predictions. Examples of predictions generated at step/operation 405 include predictions about health of a patient, predictions about likelihood of occurrence of one or more medical conditions in relation to a patient, predictions about likely effectiveness of one or more drugs in related to a patient, and/or the like. Other examples of predictions generated at step/operation 405 include predictions about response of a patient to a therapy (e.g., to a pharmaceutical), predictions about uptake or level of uptake of a therapy based on effect label (e.g., uptake of statins based on predicted levels of cholesterol), predictions about patient response to a drug (e.g., low, medium, or high degree of response), etc.

At step/operation 406, the predictive inference computing entity 106 performs one or more prediction-based actions based at least in part on the one or more predictions. In some embodiments, in response to detecting critical health conditions of a patient, the predictive inference computing entity 106 performs automated actions to address the critical health conditions of the patient. In some embodiments, in response to detecting a particular medical need of a patient, the predictive inference computing entity 106 performs automated actions to address the particular medical need of the patient. Examples of prediction-based actions include automated scheduling of medical appointments, automated physician notifications, automated patient notifications, automated generation of drug prescriptions, automated healthcare facility load balancing actions, automated addition of information to patient records, automated generation of medical information displays, and/or the like.

In some embodiments, if a prediction indicates that a patient predictive entity has a low response to a therapy (e.g., to a drug), the predictive inference computing entity 106 can cause a medical professional to run follow-up tests to confirm the existing therapy for the patient predictive entity, choose a different therapy for the patient predictive entity, change quantity of an existing therapy for the patient predictive entity, etc. For example, if an individual is predicted to respond very highly to opioids (e.g., to oxytocin, codeine, methadone, etc.), then the predictive inference computing entity 106 can cause a medical professional to either prescribe alternative pain medications for the individual or lower the prescribed opioid dosages for the individual.

In some embodiments, a prediction-based action may include determining conclusions about particular biological conditions based on results of a cohort of patients. In some embodiments, if a prediction based on a higher-level feature (e.g., a higher-level feature related to genes, biological pathways, etc.) indicates that a patient predictive entity has a high risk of an adverse drug reaction, the predictive inference computing entity 106 may determine the predictive input features (e.g., SNPs) that are associated with the high risk for a cohort of patients and use the noted determination to infer predictive insights about genetic screening tests.

### V. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for performing predictive inference using custom-parameterized dimensionality reduction, the computer-implemented method comprising:
identifying a group of predictive input features, wherein each predictive input feature is associated with an input feature position in a predictive geometric spectrum;
identifying one or more predictive markers, wherein each predictive marker is associated with a marker position in the predictive geometric spectrum;
for each predictive marker:
determining a per-marker proximate subset of the group of predictive input features for the predictive marker based at least in part on the marker position for the predictive marker and each input feature position for a predictive input feature of the group of predictive input features,
determining, for each predictive input feature in the per-marker proximate subset, a per-feature correlation value for the predictive input feature and a target feature associated with the predictive inference, and
determining, based at least in part on each per-feature correlation value for a predictive input feature in the per-marker proximate subset, a per-marker feature for the predictive marker;
determining one or more refined features for the group of predictive input features based at least in part on each per-marker feature for a predictive marker of the one or more predictive markers;
performing the predictive inference based at least in part on the one or more refined features to generate one or more predictions; and
performing one or more prediction-based actions based at least in part on the one or more predictions.

2. The computer-implemented method of claim 1, wherein determining the per-marker proximate subset for a predictive marker of the one or more predictive markers comprises:
determining, for each predictive input feature in the group of predictive input features, an feature-marker predictive distance measure in the predictive geometric spectrum between the predictive input feature and the predictive marker associated with predictive marker; and
determining the per-marker proximate subset for the predictive marker based at least in part on each feature-marker predictive distance measure for a predictive input feature in the group of predictive input features.

3. The computer-implemented method of claim 2, wherein:
the predictive geometric spectrum defines one or more predictive spectrum units,
the one or more predictive spectrum units comprise a target predictive spectrum unit for the predictive marker, and
the feature-marker predictive distance measure for a predictive input feature in the group of predictive input features is set to a maximal value if the input feature position for the predictive input feature falls outside the target predictive spectrum unit.

4. The computer-implemented method of claim 1, wherein determining the per-feature correlation value between a predictive input feature of the group of predictive features and the target feature comprises:
determining a feature value for the predictive input feature;
determining an association value for the predictive input feature and the target feature; and
determining the per-feature correlation value based at least in part on the feature value and the association value.

5. The computer-implemented method of claim 4, wherein
the group of predictive input features comprise a group of genetic variant data objects,
the feature value for a predictive input feature in the group of predictive input features is determined based at least in part on a zygosity value for the genetic variant data object of the group of genetic variant data objects that is associated with the predictive input feature, and
the association value for a predictive input feature in the group of predictive input features is determined based at least in part on a chi-square association value for the genetic variant data object of the group of genetic variant data objects that is associated with the predictive input feature with respect to the target feature.

6. The computer-implemented method of claim 5, wherein the target feature is an ordinal categorical feature.

7. The computer-implemented method of claim 4, wherein:
the group of predictive input features comprise a group of numeric feature data objects,
the feature value for a predictive input feature in the group of predictive input features is determined based at least in part on a numeric value for the numeric feature data object of the group of numeric feature data objects that is associated with the predictive input feature, and
the association value for a predictive input feature in the group of predictive input features is determined based at least in part on a Pearson correlation value for the numeric feature data object of the group of numeric feature data objects that is associated with the predictive input feature with respect to the target feature.

8. The computer-implemented method of claim 4, wherein:
the target feature is a numeric feature, and
the association value for a predictive input feature in the group of predictive input features is determined based at least in part on a Pearson correlation value for the predictive input feature with respect to the target feature.

9. The computer-implemented method of claim 1, wherein determining the one or more refined features based at least in part on each per-marker feature for a predictive marker of the one or more predictive markers comprises:
for each predictive marker of the one or more predictive markers that is associated with one or more related predictive input features in the group of predictive input features that belong to the per-marker proximate subset for the predictive marker,
determining an investigation need indicator for the predictive marker based at least in part on the per-marker feature for the predictive marker and each per-feature correlation value for a related predictive input feature of the one or more related predictive input features;
determining whether the investigation need indicator satisfies an investigation need threshold condition; and
in response to determining that the investigation need indicator satisfies the investigation need threshold condition, performing a predictive correlation analysis on the one or more related predictive input features to determine a related subset of the one or more refined features.

10. The computer-implemented method of claim 1, wherein each predictive input feature of the group of predictive input features describes zygosity of a respective single-nucleotide polymorphism.

11. An apparatus for performing predictive inference using custom-parameterized dimensionality reduction, the apparatus comprising at least one processor and at least one memory including program code, the at least one memory and the program code configured to, with the processor, cause the apparatus to at least:
identify a group of predictive input features, wherein each predictive input feature is associated with an input feature position in a predictive geometric spectrum;
identify one or more predictive markers, wherein each predictive marker is associated with a marker position in the predictive geometric spectrum;
for each predictive marker:
determine a per-marker proximate subset of the group of predictive input features for the predictive marker based at least in part on the marker position for the predictive marker and each input feature position for a predictive input feature of the group of predictive input features,
determine, for each predictive input feature in the per-marker proximate subset, a per-feature correlation value for the predictive input feature and a target feature associated with the predictive inference, and
determine, based at least in part on each per-feature correlation value for a predictive input feature in the per-marker proximate subset, a per-marker feature for the predictive marker;
determine one or more refined features for the group of predictive input features based at least in part on each per-marker feature for a predictive marker of the one or more predictive markers;
perform the predictive inference based at least in part on the one or more refined features to generate one or more predictions; and
perform one or more prediction-based actions based at least in part on the one or more predictions.

12. The apparatus of claim 11, wherein determining the per-marker proximate subset for a predictive marker of the one or more predictive markers comprises:
determining, for each predictive input feature in the group of predictive input features, an feature-marker predictive distance measure in the predictive geometric spectrum between the predictive input feature and the predictive marker associated with predictive marker; and
determining the per-marker proximate subset for the predictive marker based at least in part on each feature-marker predictive distance measure for a predictive input feature in the group of predictive input features.

13. The apparatus of claim 12, wherein:
the predictive geometric spectrum defines one or more predictive spectrum units,
the one or more predictive spectrum units comprise a target predictive spectrum unit for the predictive marker, and
the feature-marker predictive distance measure for a predictive input feature in the group of predictive input features is set to a maximal value if the input feature position for the predictive input feature falls outside the target predictive spectrum unit.

14. The apparatus of claim 11, wherein determining the per-feature correlation value between a predictive input feature of the group of predictive features and the target feature comprises:
determining a feature value for the predictive input feature;
determining an association value for the predictive input feature and the target feature; and
determining the per-feature correlation value based at least in part on the feature value and the association value.

15. The apparatus of claim 14, wherein
the group of predictive input features comprise a group of genetic variant data objects,
the feature value for a predictive input feature in the group of predictive input features is determined based at least in part on a zygosity value for the genetic variant data object of the group of genetic variant data objects that is associated with the predictive input feature, and
the association value for a predictive input feature in the group of predictive input features is determined based at least in part on a chi-square association value for the genetic variant data object of the group of genetic variant data objects that is associated with the predictive input feature with respect to the target feature.

16. The apparatus of claim 14, wherein:
the group of predictive input features comprise a group of numeric feature data objects,
the feature value for a predictive input feature in the group of predictive input features is determined based at least in part on a numeric value for the numeric feature data object of the group of numeric feature data objects that is associated with the predictive input feature, and
the association value for a predictive input feature in the group of predictive input features is determined based at least in part on a Pearson correlation value for the numeric feature data object of the group of numeric feature data objects that is associated with the predictive input feature with respect to the target feature.

17. The apparatus of claim 11, wherein determining the one or more refined features based at least in part on each per-marker feature for a predictive marker of the one or more predictive markers comprises:
for each predictive marker of the one or more predictive markers that is associated with one or more related predictive input features in the group of predictive input features that belong to the per-marker proximate subset for the predictive marker,
determining an investigation need indicator for the predictive marker based at least in part on the per-marker feature for the predictive marker and each per-feature correlation value for a related predictive input feature of the one or more related predictive input features;
determining whether the investigation need indicator satisfies an investigation need threshold condition; and
in response to determining that the investigation need indicator satisfies the investigation need threshold condition, performing a predictive correlation analysis on the one or more related predictive input features to determine a related subset of the one or more refined features.

18. A computer program product for predictive data analysis using hybrid document embedding, the computer program product comprising at least one non-transitory computer-readable storage medium having computer-readable program code portions stored therein, the computer-readable program code portions configured to:
identify a group of predictive input features, wherein each predictive input feature is associated with an input feature position in a predictive geometric spectrum;
identify one or more predictive markers, wherein each predictive marker is associated with a marker position in the predictive geometric spectrum;
for each predictive marker:
determine a per-marker proximate subset of the group of predictive input features for the predictive marker based at least in part on the marker position for the predictive marker and each input feature position for a predictive input feature of the group of predictive input features,
determine, for each predictive input feature in the per-marker proximate subset, a per-feature correlation value for the predictive input feature and a target feature associated with the predictive inference, and
determine, based at least in part on each per-feature correlation value for a predictive input feature in the per-marker proximate subset, a per-marker feature for the predictive marker;
determine one or more refined features for the group of predictive input features based at least in part on each per-marker feature for a predictive marker of the one or more predictive markers;
perform the predictive inference based at least in part on the one or more refined features to generate one or more predictions; and
perform one or more prediction-based actions based at least in part on the one or more predictions.

19. The computer program product of claim 18, wherein:
the predictive geometric spectrum defines one or more predictive spectrum units,
the one or more predictive spectrum units comprise a target predictive spectrum unit for the predictive marker, and
the feature-marker predictive distance measure for a predictive input feature in the group of predictive input features is set to a maximal value if the input feature position for the predictive input feature falls outside the target predictive spectrum unit.

20. The computer program product of claim 18, wherein determining the one or more refined features based at least in part on each per-marker feature for a predictive marker of the one or more predictive markers comprises:
for each predictive marker of the one or more predictive markers that is associated with one or more related predictive input features in the group of predictive input features that belong to the per-marker proximate subset for the predictive marker,
determining an investigation need indicator for the predictive marker based at least in part on the per-marker feature for the predictive marker and each per-feature correlation value for a related predictive input feature of the one or more related predictive input features;
determining whether the investigation need indicator satisfies an investigation need threshold condition; and
in response to determining that the investigation need indicator satisfies the investigation need threshold condition, performing a predictive correlation analysis on the one or more related predictive input features to determine a related subset of the one or more refined features.
